(19) United States
(12) Patent Application Publication
(10) Pub. No.: US 2026/0263448 A1
(43) Pub. Date: Sep. 10, 2026

(54) CONTROLLED RELEASE INJECTABLE CARIPRAZINE FORMULATION

(71) Applicant: Richter Gedeon Nyrt., Budapest (HU)

(72) Inventors: Melinda Konta, Budapest (HU); József Lázár, Budapest (HU)

(21) Appl. No.: 19/165,780

(22) PCT Filed: Jul. 6, 2022
(86) PCT No.: PCT/IB2022/056227

§ 371 Date: Sep. 16, 2025

(30) Foreign Application Priority Data
Jul. 7, 2021 (HU) P2L00259

(51) Int. Cl. A61K 31/495; A61K 9/00; A61K 9/10; A61K 9/16; A61K 9/19; A61K 47/02; A61K 47/26; A61K 47/38

(52) U.S. Cl. CPC A61K 31/495; A61K 9/0019; A61K 9/10; A61K 9/1623; A61K 9/1652; A61K 9/19; A61K 47/02; A61K 47/26; A61K 47/38

(57) ABSTRACT

The present invention relates to controlled release injectable Cariprazine (trans-N-{4-[2-[4-(2,3-dichlorophenyl)-piperazin-1-yl]-ethyl]-cyclohexyl}-N′,N′-dimethylurea) formulations, which release Cariprazine over at least a one-week period, process for the preparation thereof, and the use of said formulations in the treatment and/or prevention of pathological conditions which require the modulation of dopamine receptors.

### FIELD OF THE INVENTION

The present invention relates to controlled release injectable Cariprazine (trans-N-{4-[2-[4-(2,3-dichlorophenyl)-piperazin-1-yl]-ethyl]-cyclohexyl}-N′,N′-dimethylurea) formulations, which release Cariprazine over at least a one-week period, process for the preparation thereof, and the use of said formulations in the treatment and/or prevention of pathological conditions which require the modulation of dopamine receptors.

### BACKGROUND OF THE INVENTION

Cariprazine is a dopamine D₃ receptor and D₂ receptor partial agonist with high selectivity for the D₃ receptor. WO 2005/012266 A1 discloses Cariprazine and its use for the treatment and/or prevention of pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism.

The current form of administration of Cariprazine is an immediate release capsule, sold under the brand name Vraylar in the United States of America for the treatment of schizophrenia in adults, acute treatment of manic or mixed episodes associated with bipolar I disorder in adults, and the treatment of depressive episodes associated with bipolar I disorder (bipolar depression) in adults, and in the European Union under the brand name Reagila for treatment of schizophrenia in adults. The capsules must be administered daily to patients in need thereof, due to the fast release character of the composition. For the purpose of producing a therapeutic effect in a patient, the total daily dose of Cariprazine ranges from about 0.5 mg to about 6 mg. However, inter-individual differences and co-medication may necessitate dose titrating in patients.

The longer the treatment, the higher the need for a more convenient dosage regimen, because the efficacy of a long-term therapy is closely related, among other factors, to the patients' compliance. Therefore, there is a need to provide a pharmaceutical composition, which can be administered less frequently, for example as a controlled release injection. This form of antipsychotics aims to improve medication compliance for a better therapeutic alternative and treat patients who lack insight or adhere poorly to the currently available oral medication. Moreover, the long-acting antipsychotics facilitate the use of the lowest effective dose principle, thereby reducing the probability of medication overdose and the frequency of adverse events like akathisia, dysphoria and antipsychotic-induced deficit syndrome, and thus enhancing the patient's quality of life. The controlled release injections comprising antipsychotics eliminate the need for daily medication and regular monitoring of patients; these drugs are therefore widely accepted for their convenience for all involved in the treatment of the related mental disorders (E J Park et al. Arch. Pharm. Res. 36 (2013) 651-659).

Several long acting injections are clinically available for the treatment of psychotic disorders; including haloperidol decanoate (Haldol Decanoate) and fluphenazine decanoate (Prolixin Decanoate) both dissolved in sesame oil, risperidone encapsulated into biodegradable microspheres using poly(D,L-lactide-co-glycolide) (Risperdal Consta), microcrystalline salt of olanzapine and pamoic acid (Zyprexa Relprevv/Zypadhera), paliperidone palmitate nanocrystals (Invega Trinza/Invega Sustenna) and aripiprazole monohydrate crystals (Abilify Maintena) suspended in aqueous solution.

GB 2054371 A discloses pharmaceutical compositions for parenteral administration, comprising a decanoic acid ester of haloperidol in sesame oil as a solvent.

WO 95/13814 A1 discloses biodegradable and biocompatible microparticles containing risperidone, which are capable of releasing the active pharmaceutical ingredient over an extended period of time.

WO 97/44039 A1 discloses a pharmaceutical composition suitable as a depot formulation for administration via intramuscular or subcutaneous injection, comprising paliperidone palmitate ester suspended in water.

WO 99/25354 A2 discloses depot injections for administration via intramuscular or subcutaneous route, comprising paliperidone palmitate with an effective average particle size of less than 2000 nm suspended in water.

WO 99/16313 A1 discloses a formulation comprising olanzapine pamoate salt and an oleaginous or cholesterol microsphere carrier.

WO 03/007912 A2 discloses an amorphous, lyophilized parenteral formulation comprising olanzapine as an active ingredient, a solubilizer and lactose as a stabilizer.

WO 2005/041937 A2 discloses long acting injections comprising aripiprazole, which upon injection release aripiprazole over a period of at least two weeks. The formulation comprises aripiprazole, suspending agents, and optionally bulking agents, buffering agents, and pH-adjusting agents.

WO 2017/119936 A1 discloses long acting injectable formulations comprising a poorly water-soluble active pharmaceutical ingredient, for example cariprazine and a non-aqueous liquid vehicle, wherein the active pharmaceutical ingredient is dispersed as discrete particles having a d₉₀ particle size of about 0.5 μm to about 25 μm in the formulation, and wherein the formulation is non-gelling and thixotropic with a viscosity of less than 10 poise at a shear rate of 10/s at 25° C.

CN 108261394 A discloses an injection comprising Cariprazine hydrochloride, which releases the active ingredient continuously for at least one week. The in vivo tested composition comprises Cariprazine hydrochloride with a volume mean diameter of 3.221 μm, Tween 20, Span 20 and water for injections. The release of the active ingredient from the exemplary formulation appears to be too rapid, resulting in relatively high initial plasma levels, which should be improved upon in a truly effective depot formulation.

WO 2021/119166 A1 discloses pharmaceutical compositions in the form of a microparticle, a microsphere, a nanoparticle, or a combination thereof comprising cariprazine ionically complexed with a biodegradable and biocompatible polymer.

Consequently, there is a need to provide a safe controlled release injectable Cariprazine formulation, which releases the desired substantially constant concentrations of Cariprazine over at least a one-week period with negligible chance of burst effect of the active ingredient.

### SUMMARY OF THE INVENTION

The present invention provides a controlled release injectable formulation in the form of a suspension which upon injection releases Cariprazine over a period of at least one week, which comprises: Cariprazine base, and one or more buffering agents, and optionally one or more bulking agents, and optionally one or more wetting agents, and optionally one or more viscosity increasing agents, and optionally one or more pH-adjusting agents, and an aqueous vehicle therefor.

In one embodiment the present invention provides a freeze-dried controlled release injectable formulation, which comprises: Cariprazine base, one or more bulking agents, and/or one or more buffering agents, and optionally one or more wetting agents, and optionally one or more viscosity increasing agents, and optionally one or more pH-adjusting agents, which formulation upon constitution with an aqueous reconstitution medium forms an injectable formulation, which upon injection releases Cariprazine over a period of at least one week.

In another embodiment, the present invention provides a controlled release Cariprazine injectable formulation as defined above for use in the treatment and/or prevention of pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism.

In yet another embodiment, the present invention provides the use of a controlled release Cariprazine injectable formulation as defined above in the manufacture of a medicament for the treatment and/or prevention of pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism.

In a further embodiment, the present invention provides a method of treating a patient suffering from pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism, wherein the method comprises the administration of the controlled release Cariprazine injectable formulation as defined above, less frequent than daily to a patient in need thereof.

### BRIEF DESCRIPTION OF THE DRAWINGS

FIG. 1 illustrates the mean plasma concentrations vs. time profiles of Cariprazine in rats after administration of the formulations according to Example 1 and the Reference example.

FIG. 2A illustrates the mean plasma concentrations vs. time profiles of Cariprazine in rats after intramuscular and subcutaneous administration of the formulation according to Example 2A.

FIG. 2B illustrates the mean plasma concentrations vs. time profiles of Cariprazine in rats after intramuscular administration of the formulation according to Example 2B.

FIG. 3 illustrates the mean plasma concentrations vs. time profiles of Cariprazine in rats after subcutaneous administration of the formulations according to Examples 3A and 3B.

### DETAILED DESCRIPTION OF THE INVENTION

The present invention provides a controlled release injectable formulation in the form of a suspension which upon injection releases Cariprazine over a period of at least one week, which comprises: Cariprazine base, and one or more buffering agents, optionally one or more bulking agents, and optionally one or more wetting agents, and optionally one or more viscosity increasing agents, and optionally one or more pH-adjusting agents, and an aqueous vehicle therefor.

The present invention also provides a controlled release injectable formulation in the form of a suspension which upon injection releases Cariprazine over a period of at least one week, consisting of: Cariprazine base, and one or more buffering agents, optionally one or more bulking agents, and optionally one or more wetting agents, and optionally one or more viscosity increasing agents, and optionally one or more pH-adjusting agents, and an aqueous vehicle therefor.

The present invention also provides a freeze-dried controlled release injectable formulation, which comprises: Cariprazine base, one or more bulking agents, and/or one or more buffering agents, and optionally one or more wetting agents, and optionally one or more viscosity increasing agents, and optionally one or more pH-adjusting agents, which formulation upon constitution with an aqueous reconstitution medium forms an injectable formulation, which upon injection releases Cariprazine over a period of at least one week.

The present invention also provides a freeze-dried controlled release injectable formulation, consisting of: Cariprazine base, one or more bulking agents, and/or one or more buffering agents, and optionally one or more wetting agents, and optionally one or more viscosity increasing agents, and optionally one or more pH-adjusting agents, which formulation upon constitution with an aqueous reconstitution medium forms an injectable formulation, which upon injection releases Cariprazine over a period of at least one week.

In order to achieve a controlled release profile for acidic and basic drugs, the most obvious and commonly used method is the formation of an insoluble salt, thus controlling the solubility and dissolution. Moreover, it is required in clinical practice, to restrict the pH of injections to a range that mimics the physiological values. The target pH for maximum physiological acceptability is approximately pH 7.4. In practice, however, a reasonably wide pH range can be tolerated, for example, via intramuscular route pH ranges of 3 to 11 and via subcutaneous route pH ranges of 3 to 6 are recommended. Vast majority of licensed products have a pH between 3 and 9. A pH outside this range should be avoided, if possible, since a pH of greater than 9 can cause tissue necrosis, whereas a pH of less than 3 may cause pain and phlebitis. (Gibson M, Pharmaceutical preformulation and formulation (2009) p. 326-27).

Based on the solubility of Cariprazine hydrochloride in the pH-range of human muscle and subcutaneous tissues, namely at pH 6.8-7.5 it is expected to be suitable for a depot formulation, as it is practically insoluble in that environment.

TABLE 1
The solubility of Cariprazine hydrochloride
at different pH values (37° C.)

| | Solubility | |
|---|---|---|
| pH | (average, mg/ml) | Solubility category (USP) |
| pH = 3 | 6.0 | Slightly soluble |
| pH = 4.5 | 13.8 | Sparingly soluble |
| pH = 5.5 | 0.27 | Very slightly soluble |
| pH = 6.8 | 0.005 | Practically insoluble |
| pH = 7.5 | 0.001 | Practically insoluble |

However, during the in vivo pharmacokinetic studies, it has unexpectedly been found, that the release of Cariprazine hydrochloride from the depot injection formulation is remarkably high, even in case of a relatively large volume mean diameter of the active ingredient. This might result in the so called “burst effect” inducing for example akathisia—persistent motor restlessness and muscle tightness—as a serious side effect.

The term “burst effect” as used herein refers to the phenomenon, when a larger amount of the active pharmaceutical ingredient is released initially within a short timeframe upon administration of a controlled release injection causing significant side effects, for example extrapyramidal symptoms as severe akathisia.

Therefore, the aim of the present invention is to provide a safe controlled release injection formulation comprising Cariprazine, wherein the plasma concentrations of Cariprazine increase slowly after the administration of the injection, without remarkable burst-effect and the slow depot release maintains the Cariprazine concentration within the same range as of the oral formulation.

In order to achieve the desired controlled release profile, several different formulations were tested, and it has been found, that in contrast to the Cariprazine hydrochloride salt, the Cariprazine base is suitable for a controlled release injectable formulation, without the use of release modifying polymers, despite the fact that the solubility of Cariprazine base and the hydrochloride salt is similarly practically insoluble in the pH-range of human muscle and subcutaneous tissues:

TABLE 2
The solubility of Cariprazine base at different pH values (37° C.)

| | Solubility | |
|---|---|---|
| pH | (average, mg/ml) | Solubility category (USP) |
| pH = 3 | 0.204 | Very slightly soluble |
| pH = 4.4 | 0.357 | Very slightly soluble |
| pH = 6.8 | 0.0007 | Practically insoluble |
| pH = 7.4 | not detectable | Practically insoluble |

The omission of release modifying polymers is advantageous, as, inter alia, the presence of residual organic solvents in the final formulation and other side effects caused by the polymers is avoided.

The controlled release injectable formulation of the present invention comprises Cariprazine base, one or more buffering agents, and optionally one or more bulking agents, and optionally one or more wetting agents, and optionally one or more viscosity increasing agents, and optionally one or more pH-adjusting agents, and an aqueous vehicle therefor, when it is in the form a suspension; and comprises Cariprazine base, one or more bulking agents, and/or one or more buffering agents, and optionally one or more wetting agents, and optionally one or more viscosity increasing agents, and optionally one or more pH-adjusting agents, when it is in the form of a freeze dried formulation.

It has surprisingly been found, that the controlled release formulations of the present invention are capable of maintaining a substantially constant Cariprazine drug plasma concentration with negligible chance of burst effect when administered by injection. Moreover, the controlled release formulation of the present invention provides a constant Cariprazine drug plasma concentration over at least a one-week period.

The daily starting dose for the orally administered immediate release Cariprazine capsules is about 1.5 mg for adults, and the total daily dose is from about 1.5 mg to about 6 mg. Thus, to administer a drug dose equivalent to one to more than eight weeks of the oral dosage quantity requires the administration of a very large amount of Cariprazine as a single dose. However, the controlled release Cariprazine injectable formulation of the present invention may be administered to deliver the desired amount of the drug with negligible chance of burst effect.

The controlled release Cariprazine injectable formulation of the invention can be administered as a ready-to-use suspension, or by freeze-drying this suspension, and thus obtaining a solid product, which can be later reconstituted to its original form for injection.

The desired controlled release period is at least about two weeks, up to eight weeks or more, preferably about three to about four weeks. It has also been found, that by selecting a specific volume mean diameter of the Cariprazine base an injectable formulation having the desired controlled release properties of the active pharmaceutical ingredient can be achieved. The formulations comprising Cariprazine base characterized by a volume mean diameter within from about 1 μm to about 250 μm show superior pharmacokinetic profiles, exceptional tolerability and good injectability. Therefore, in order to achieve the desired controlled release, the Cariprazine base preferably has a volume mean diameter within the range from about 1 μm to about 250 μm, preferably from about 3 μm to about 100 μm, and more preferably from about 3 μm to about 50 μm.

The reduction of the volume mean diameter of Cariprazine base can be carried out by methods well known to those of ordinary skill in the art, for example by employing a milling procedure or by crystallization.

The term “volume mean diameter” (VMD) refers to the average particle diameter by volume as measured by laser-light scattering (LLS) methods. Particle size distribution is measured by LLS methods and volume mean diameter is calculated from the particle size distribution.

The term “d₁₀” refers to the particle diameter corresponding to 10% cumulative (from 0 to 100%) undersize particle size distribution, i.e. the diameter at which 10% of the sample's mass is comprised of particles with a diameter less than this value.

The term “d₅₀” refers to the particle diameter corresponding to 50% cumulative (from 0 to 100%) undersize particle size distribution, i.e. the diameter at which 50% of the sample's mass is comprised of particles with a diameter less than this value.

The term “d₉₀” refers to the particle diameter corresponding to 90% cumulative (from 0 to 100%) undersize particle size distribution, i.e. the diameter at which 90% of the sample's mass is comprised of particles with a diameter less than this value.

In one embodiment the controlled release injectable formulation of the invention comprises: Cariprazine base, one or more buffering agents, and optionally one or more bulking agents, and optionally one or more wetting agents, and optionally one or more viscosity increasing agents, and optionally one or more pH-adjusting agents, and an aqueous vehicle therefor.

In another embodiment the controlled release injectable formulation of the invention consist of: Cariprazine base, one or more buffering agents, and optionally one or more bulking agents, and optionally one or more wetting agents, and optionally one or more viscosity increasing agents, and optionally one or more pH-adjusting agents, and an aqueous vehicle therefor.

The formulation comprises the buffering agent from about 0.1 to about 10% by weight, or from about 1 to about 6% by weight, or from about 1 to about 4% by weight based on the total weight of the injectable formulation. Examples of buffering agents suitable for use in the formulations include, but are not limited to, one or more of the following: acetic acid, ammonium phosphate, ammonium sulfate, L-arginine, asparagine, boric acid, calcium carbonate, calcium lactate, tribasic calcium phosphate, citric acid monohydrate, dibasic sodium phosphate, diethanolamine, glutamic acid, glycine, histidine, lysine acetate, maleic acid, malic acid, methionine, monobasic sodium phosphate, monoethanolamine, monosodium glutamate, potassium citrate, sodium acetate, sodium bicarbonate, sodium borate, sodium carbonate, sodium citrate dihydrate, sodium lactate, sodium tartarate; sodium succinate, phosphoric acid, triethanolamine, tromethamine (Tris).

The most preferred buffering agents are the following: acetic acid, monobasic sodium phosphate, phosphoric acid, sodium citrate, histidine, tribasic calcium phosphate, tromethamine (Tris).

The formulation comprises the bulking agent from about 0 to about 90% by weight, or from about 5 to about 60% by weight, or from about 10 to about 40% by weight based on the total weight of the injectable formulation. Examples of bulking agents suitable for use in the formulations include, but are not limited to, one or more of the following: dextran 40, dextrose, glucose, glycine, histidine, inositol, lactose, mannitol, powdered cellulose, raffinose, sorbitol, sucrose, trehalose.

The most preferred bulking agents are the following: mannitol, powdered cellulose, sucrose, trehalose.

The formulation comprises the wetting agent from about 0 to about 25% by weight, or from about 0.1 to about 15% by weight, or from about 1 to about 10% by weight based on the total weight of the injectable formulation. Examples of wetting agents suitable for use in the formulations include, but are not limited to, one or more of the following: alpha tocopherol, benzalkonium chloride, benzethonium chloride, cetrimide, cetrimonium bromide, glyceryl monooleate, glyceryl tricaprylate, lecithin, myristyl alcohol, phospholipids, polyoxyethylene alkyl ethers, polyoxyethylene castor oil derivatives, poloxamer, polyoxyethylene sorbitan fatty acid esters, polyoxyethylene stearates, polyoxyl 15 hydroxystearate, sorbitan esters (sorbitan fatty acid esters), sucrose palmitate, sucrose stearate, vitamin E polyethylene glycol succinate.

The most preferred wetting agents are the following: alpha tocopherol, benzalkonium chloride, lecithin, phospholipids, polyoxyethylene alkyl ethers, polyoxyethylene castor oil derivatives, poloxamer, polyoxyethylene sorbitan fatty acid esters.

The formulation comprises the viscosity increasing agent from about 0 to about 40% by weight, or from about 1 to about 20% by weight or from about 2 to about 10% by weight based on the total weight of the injectable formulation. Examples of viscosity increasing agents suitable for use in the formulations include, but are not limited to, one or more of the following: agar, alginic acid, ammonium sulfate, betadex sulfobutyl ether sodium, carboxymethylcellulose calcium, carboxymethylcellulose sodium, cellulose microcrystalline and carboxymethylcellulose sodium coprocessed, ceratonia, cetostearyl alcohol, chitosan, ethylcellulose, glycerin, glyceryl behenate, hectorite, hydroxyethyl cellulose, hydroxyethylmethyl cellulose, hydroxypropyl cellulose, hydroxypropyl starch, methylcellulose, myristyl alcohol, polydextrose, poly(methylvinyl ether/maleic anhydride), polyvinyl alcohol, propylene glycol alginate, saponite, sodium alginate, starch, stearyl alcohol, sucrose, tragacanth, xanthan gum.

The most preferred viscosity increasing agents are the following: carboxymethylcellulose sodium, glycerin, hydroxyethyl cellulose, hydroxyethylmethyl cellulose, hydroxypropyl cellulose, hydroxypropyl starch, polyvinyl alcohol.

The formulation comprises the pH-adjusting agent from about 0 to about 5% by weight, or from about 0.1 to about 3% by weight, or from about 0.5 to about 1% by weight based on the total weight of the injectable formulation. Examples of pH-adjusting agents suitable for use in the formulations include, but are not limited to, one or more of the following: acetic acid, ammonia solution, ammonium hydroxide, calcium hydroxide, boric acid, hydrochloric acid, phosphoric acid, sodium hydroxide.

The most preferred pH-adjusting agents are the following: acetic acid, hydrochloric acid, sodium hydroxide.

The aqueous vehicle is preferably water for injections.

The materials for the present invention can be purchased or prepared by a variety of procedures well known to those of ordinary skill in the art.

In one embodiment the controlled release injectable formulation of the invention is in the form of a suspension, where:
a) the Cariprazine base is present in an amount within the range from about 0.5 to about 25 weight/volume %,
b) the bulking agent is present in an amount within the range from about 0 to about 60 weight/volume %,
c) the buffering agent is present in an amount within the range from about 0 to about 5 weight/volume %
d) the wetting agent is present in an amount within the range from about 0 to about 5 weight/volume %
e) the viscosity increasing agent is present in an amount within the range from about 0 to about 15 weight/volume %
f) the pH-adjusting agent is present in an amount within the range from about 0 to about weight/volume 3%
   based on the total volume of suspension,
g) and water for injections in an amount sufficient to reach the final desired volume of 0.5 to 4 ml of the injection.

In a further embodiment the controlled release injectable formulation of the invention is a suspension containing Cariprazine base having a volume mean diameter within the range from about 1 μm to about 250 μm.

In a further embodiment the controlled release injectable formulation of the invention is in the form of a suspension containing Cariprazine base having a volume mean diameter within the range from about 2 μm to about 200 μm.

In a further embodiment the controlled release injectable formulation of the invention is in the form of a suspension containing Cariprazine base having a volume mean diameter within the range from about 3 μm to about 150 μm.

In a further embodiment the controlled release injectable formulation of the invention is in the form of a suspension containing Cariprazine base having a volume mean diameter within the range from about 3 μm to about 100 μm.

In a further embodiment suspension containing Cariprazine base having a volume mean diameter within the range from about 3 μm to about 50 μm.

In a further embodiment the controlled release injectable formulation of the invention is in the form of a suspension containing Cariprazine base having a volume mean diameter within the range from about 3 μm to about 25 μm.

In a further embodiment the controlled release injectable formulation of the invention is in the form of a suspension containing Cariprazine base having a volume mean diameter within the range from about 5 μm to about 25 μm.

In a further embodiment the controlled release Cariprazine injectable formulation of the invention can be administered by freeze-drying the ready-to-use suspension obtaining a solid product, which can be later reconstituted to its original form for injection.

The freeze-dried injection can be formulated by a method known per se in the art.

The freeze-dried formulation is preferably reconstitutable in from about 0.5 to about 5 ml of a suitable aqueous reconstitution medium, preferably 2 ml, to obtain solutions having concentrations of Cariprazine base approximately between 5 and 60 mg/ml, and can be dissolved in a pharmaceutically acceptable carrier, such as water for injections or water for injections containing excipients, such as bulking agents, buffering agents, wetting agents, viscosity increasing agents and pH-adjusting agents.

For example, for an intramuscular or subcutaneous injection, the lyophilized plug may be dissolved in 2 ml of reconstitution medium.

In a further embodiment the controlled release injectable formulation of the invention is in the form of a freeze-dried formulation containing Cariprazine base having a volume mean diameter within the range from about 1 μm to about 250 μm.

In a further embodiment the controlled release injectable formulation of the invention is in the form of a freeze-dried formulation containing Cariprazine base having a volume mean diameter within the range from about 2 μm to about 200 μm.

In a further embodiment the controlled release injectable formulation of the invention is in the form of a freeze-dried formulation containing Cariprazine base having a volume mean diameter within the range from about 3 μm to about 150 μm.

In a further embodiment the controlled release injectable formulation of the invention is in the form of a freeze-dried formulation containing Cariprazine base having a volume mean diameter within the range from about 3 μm to about 100 μm.

In a further embodiment the controlled release injectable formulation of the invention is in the form of a freeze-dried formulation containing Cariprazine base having a volume mean diameter within the range from about 3 μm to about 50 μm.

In a further embodiment the controlled release injectable formulation of the invention is in the form of a freeze-dried formulation containing Cariprazine base having a volume mean diameter within the range from about 3 μm to about 25 μm.

In a further embodiment the controlled release injectable formulation of the invention is in the form of a freeze-dried formulation containing Cariprazine base having a volume mean diameter within the range from about 5 μm to about 25 μm.

In one embodiment the present invention provides a controlled release injectable formulation for use in the treatment and/or prevention of pathological conditions which require the modulation of dopamine receptors, selected from the group psychoses (including schizophrenia, and schizo-affective disorders), drug abuse, cognitive impairment accompanying schizophrenia (including positive symptoms, negative symptoms, and cognitive symptoms), mild-to-moderate cognitive deficits, dementia, psychotic states associated with dementia, eating disorders, attention deficit disorders, hyperactivity disorders in children, psychotic depression, mania, paranoid and delusional disorders, dyskinetic disorders, anxiety, sexual dysfunction, sleep disorders, emesis, aggression and autism.

In a further embodiment the present invention provides a controlled release injectable formulation for use in the treatment and/or prevention of schizophrenia, and/or mania, and/or depression, and/or autism.

In one embodiment the present invention provides a controlled release injectable formulation in the manufacture of a medicament for the treatment and/or prevention of pathological conditions which require the modulation of dopamine receptors selected from the group of psychoses (including schizophrenia and schizo-affective disorders), drug abuse, cognitive impairment accompanying schizophrenia (including positive symptoms, negative symptoms, and cognitive symptoms), mild-to-moderate cognitive deficits, dementia, psychotic states associated with dementia, eating disorders, attention deficit disorders, hyperactivity disorders in children, psychotic depression, mania, paranoid and delusional disorders, dyskinetic disorders, anxiety, sexual dysfunction, sleep disorders, emesis, aggression and autism.

In a further embodiment the present invention provides a controlled release injectable formulation in the manufacture of a medicament for the treatment and/or prevention of schizophrenia, and/or mania, and/or depression, and/or autism.

In one embodiment the present invention is directed to the method of treating a patient suffering from pathological conditions which require the modulation of dopamine receptors, selected from the group of psychoses (including schizophrenia and schizo-affective disorders), drug abuse, cognitive impairment accompanying schizophrenia (including positive symptoms, negative symptoms, and cognitive symptoms), mild-to-moderate cognitive deficits, dementia, psychotic states associated with dementia, eating disorders, attention deficit disorders, hyperactivity disorders in children, psychotic depression, mania, paranoid and delusional disorders, dyskinetic disorders, anxiety, sexual dysfunction, sleep disorders, emesis, aggression and autism.

In a further embodiment the present invention is directed to the method of treating a patient suffering from schizophrenia, and/or mania, and/or depression, and/or autism.

In one embodiment, the present invention provides a method for preparing the controlled release Cariprazine injectable formulation described above which includes the steps of:
1) micronizing the Cariprazine base to obtain the desired particle size,
2) homogenizing the Cariprazine base with the dispersion medium by using ultrasound and/or shear force (e.g. Ultra-Turrax® disperser).

As used herein, the term “aqueous vehicle” refers to a water-based carrying agent in which the ingredients of a pharmaceutical formulation are dissolved or dispersed.

As used herein, the term “aqueous reconstitution medium” refers to water or a water-based solution, which is used for redissolving or redispersing (e.g. resuspending) the ingredients of a pharmaceutical formulation before administration to a patient.

As used herein, the term “treating” includes prophylaxis of the named condition or amelioration or elimination of the condition once it has been established.

As used herein, the term “modulating” or “modulate” refers to an effect of altering a biological activity, especially a biological activity associated with an injection site reaction.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### EXAMPLES

### Example 1: Preparation of Controlled Release Injectable Formulations Comprising Cariprazine Base for In Vivo Pharmacokinetic Studies

In order to find the optimal particle size, three different controlled release formulations were prepared by suspending Cariprazine base in a phosphate buffer. The phosphate buffer, pH=7.4 was prepared by adding 250 ml of 0.2 M potassium dihydrogen phosphate to 199.5 ml of 0.1 M sodium hydroxide and diluting the mixture to 1000 ml with purified water.

TABLE 3
Composition of the formulations of Examples 1A, 1B and 1C

| Ingredients | Quantity |
|---|---|
| Micronized Cariprazine base | 60 mg |
| Phosphate buffer, pH = 7.4 | 1 ml |

### Particle Size Distribution of Cariprazine Base in the Formulations of Example 1:

TABLE 4
Particle size distribution of the formulations
of Examples 1A, 1B and 1C

Particle size distribution (μm)

| | d₁₀ | d₅₀ | d₉₀ | VMD |
|---|---|---|---|---|
| Example 1A | 62.0 | 203 | 389 | 215 |
| Example 1B | 2.0 | 3.6 | 18.4 | 7.6 |
| Example 1C | 2.7 | 30.6 | 88.2 | 38.3 |

### Reference Example: Preparation of a Controlled Release Injectable Formulation Comprising Cariprazine Hydrochloride for In Vivo Pharmacokinetic Studies

The reference example was prepared by suspending Cariprazine hydrochloride in a phosphate buffer, pH=7.4 (as described in Example 1).

TABLE 5
Composition of the formulation of the Reference example

| Ingredients | Quantity |
|---|---|
| Cariprazine HCl | 65.12 mg |
| Phosphate buffer, pH = 7.4 | 1 ml |

### Particle Size Distribution of Cariprazine Hydrochloride in the Formulation of the Reference Example:

TABLE 6
Particle size distribution of the formulation
of the Reference Example

Particle size distribution (μm)

| | d₁₀ | d₅₀ | d₉₀ | VMD |
|---|---|---|---|---|
| Reference example | 6.1 | 19.5 | 44.5 | 23.2 |

### Example 2: Preparation of Controlled Release Injectable Formulations Comprising Cariprazine Base for In Vivo Pharmacokinetic Studies

The controlled release injectable formulations of examples 2A and 2B were prepared by suspending 1250 mg, or 450 mg, respectively of micronized Cariprazine base in 5 ml of the dispersion medium, which was prepared by dissolving carboxymethylcellulose sodium and polysorbate 80 in the phosphate buffer, pH=7.4 (as described in Example 1).

### Composition of the Dispersion Medium:

TABLE 7
Composition of the dispersion medium of Example 2

| Ingredients | Quantity |
|---|---|
| Carboxymethylcellulose sodium | 2000 mg |
| Polysorbate 80 | 200 mg |
| Phosphate buffer, pH = 7.4 | ad 100 ml |

### Composition of the Formulations:

TABLE 8
Composition of the formulations of Examples 2A and 2B

Quantity

| Ingredients | Example 2A | Example 2B |
|---|---|---|
| Micronized Cariprazine base | 1250 mg | 450 mg |
| Dispersion medium | 5 ml | 5 ml |

### Particle Size Distribution of Cariprazine in the Formulation of Examples 2A and 2B:

TABLE 9
Particle size distribution of the
formulations of Examples 2A and 2B

Particle size distribution (μm)

| | d₁₀ | d₅₀ | d₉₀ | VMD |
|---|---|---|---|---|
| Examples 2A and 2B | 1.6 | 5.4 | 17.5 | 8.3 |

### Example 3: Preparation of a Controlled Release Injectable Formulation Comprising Cariprazine Base for In Vivo Pharmacokinetic Studies

The controlled release injectable formulation of example 3A was prepared by suspending 1250 mg of micronized Cariprazine base in 5 ml of the dispersion medium, which was prepared by dissolving carboxymethylcellulose sodium in the phosphate buffer, pH=7.4 (as described in Example 1).

The controlled release injectable formulation of example 3B was prepared by suspending 1250 mg of micronized Cariprazine base in 5 ml of the dispersion medium, which was prepared by dissolving carboxymethylcellulose sodium and polysorbate 80 in the phosphate buffer, pH=7.4 (as described in Example 1).

### Composition of the Dispersion Medium:

TABLE 10
Composition of the dispersion medium of Example 3

Quantity

| Ingredients | Example 3A | Example 3B |
|---|---|---|
| Carboxymethylcellulose sodium | 2000 mg | 2000 mg |
| Polysorbate 80 | 0 mg | 200 mg |
| Phosphate buffer, pH = 7.4 | ad 100 ml | ad 100 ml |

### Composition of the Formulations:

TABLE 11
Composition of the formulations of Examples 3A and 3B

Quantity

| Ingredients | Example 3A | Example 3B |
|---|---|---|
| Micronized Cariprazine base | 1250 mg | 1250 mg |
| Dispersion medium | 5 ml | 5 ml |

### Particle Size Distribution of Cariprazine in the Formulation of Examples 3A and 3B:

TABLE 12
Particle size distribution of the
formulations of Examples 3A and 3B

Particle size distribution (μm)

| | d₁₀ | d₅₀ | d₉₀ | VMD |
|---|---|---|---|---|
| Examples 3A and 3B | 1.6 | 5.4 | 17.5 | 8.3 |

### Example 4: Preparation of a Controlled Release Injectable Formulation Comprising Cariprazine Base

The controlled release injectable formulations of example 4 were prepared by suspending micronized Cariprazine base in the dispersion medium, which was prepared by dissolving carboxymethylcellulose sodium, polysorbate 80 and sodium chloride (only in example 4C) in the phosphate buffer, pH=7.4 (as described in Example 1).

### Composition of the Dispersion Medium:

TABLE 13
Composition of the dispersion medium
of Examples 4A, 4B and 4C

| | Quantity | | |
|---|---|---|---|
| | Example | Example | Example |
| Ingredients | 4A | 4B | 4C |
| Carboxymethylcellulose sodium | 2000 mg | 2000 mg | 2000 mg |
| Polysorbate 80 | 50 mg | 90 mg | 80 mg |
| Sodium chloride | — | — | 400 mg |
| Phosphate buffer, pH = 7.4 | ad 100 ml | ad 100 ml | ad 100 ml |

### Composition of the Formulations:

TABLE 14
Composition of the formulations of Examples 4A, 4B, and 4C

| | Quantity | | |
|---|---|---|---|
| Ingredients | Example 4A | Example 4B | Example 4C |
| Micronized Cariprazine base | 625 mg | 625 mg | 120 mg |
| Dispersion medium | 2.5 ml | 2.5 ml | 2 ml |

### Particle Size Distribution of Cariprazine in the Formulation of Examples 4A, 4B and 4C:

TABLE 15
Particle size distribution of the formulations
of Examples 4A, 4B and 4C

Particle size distribution (μm)

| | d₁₀ | d₅₀ | d₉₀ |
|---|---|---|---|
| Examples 4A, 4B and 4C | 2.3 | 12.5 | 60.2 |

### Example 5: Preparation of a Lyophilized Controlled Release Injectable Formulation Comprising Cariprazine Base

The controlled release injectable formulations of Example 5 were prepared by suspending 120 mg of micronized Cariprazine base in 2 ml of the dispersion medium, which was prepared by dissolving carboxymethylcellulose sodium, polysorbate 80 and mannitol in the phosphate buffer, pH=7.4 (as described in Example 1).

The above suspension was filled into vials and transferred to a freeze dryer and lyophilized according to the following cycle:
(a) thermal treatment: freeze product at −50° C. over 0.1 h-1 h 50 min and keep at −50° C. for at least 2 h,
(b) primary drying: lower chamber pressure to approximately 50 microBar and increase product temperature to −25° C. over approximately 10 min-2 h; continue primary drying at −25° C. and 50 microBar for at least 16 h 30 min,
(c) secondary drying: increase chamber pressure to approximately 1000 microBar and increase product temperature to 35° C. over approximately 2 h; continue primary drying at 35° C. and 1000 microBar for at least 5 h,
(d) stopper the vials under-atmospheric pressure or partial vacuum using nitrogen or air and remove from the freeze dryer,
(d) seal the vials with the appropriate seals and label.

### Composition of the Dispersion Medium:

TABLE 16
Composition of the dispersion medium
of Examples 5A, 5B, and 5C

| | Quantity | | |
|---|---|---|---|
| | Example | Example | Example |
| Ingredients | 5A | 5B | 5C |
| Carboxymethylcellulose sodium | 2000 mg | 2000 mg | 2000 mg |
| Polysorbate 80 | 20 mg | 20 mg | 20 mg |
| Mannitol | 3000 mg | 4000 mg | 5000 mg |
| Phosphate buffer, pH = 7.4 | ad 100 ml | ad 100 ml | ad 100 ml |

### Composition of the Formulations:

TABLE 17
Composition of the formulations of Examples 5A, 5B and 5C

| | Quantity | | |
|---|---|---|---|
| Ingredients | Example 5A | Example 5B | Example 5C |
| Micronized Cariprazine base | 120 mg | 120 mg | 120 mg |
| Dispersion medium | 2 ml | 2 ml | 2 ml |

### Particle Size Distribution of Cariprazine in the Formulation of Examples 5A, 5B and 5C:

TABLE 18
Particle size distribution of the formulations
of Examples 5A, 5B and 5C

Particle size distribution (μm)

| | d₁₀ | d₅₀ | d₉₀ |
|---|---|---|---|
| Examples 5A, 5B and 5C | 2.3 | 12.5 | 60.2 |

### Biological Example (Animal Pharmacokinetic Data)

The controlled release formulations prepared in Example 1 and in the Reference example were injected into the thigh muscle of 6 rats at doses of 100 mg/kg. Blood samples for the evaluation of systemic exposure were collected after 0, 0.5, 1, 4, 8, 24, 48, 72, 96, 168, 336, 504, and 672 hours of administration of the controlled release formulation and analyzed for Cariprazine. FIG. 1 shows the mean plasma concentrations vs. time profiles of Cariprazine in rats.

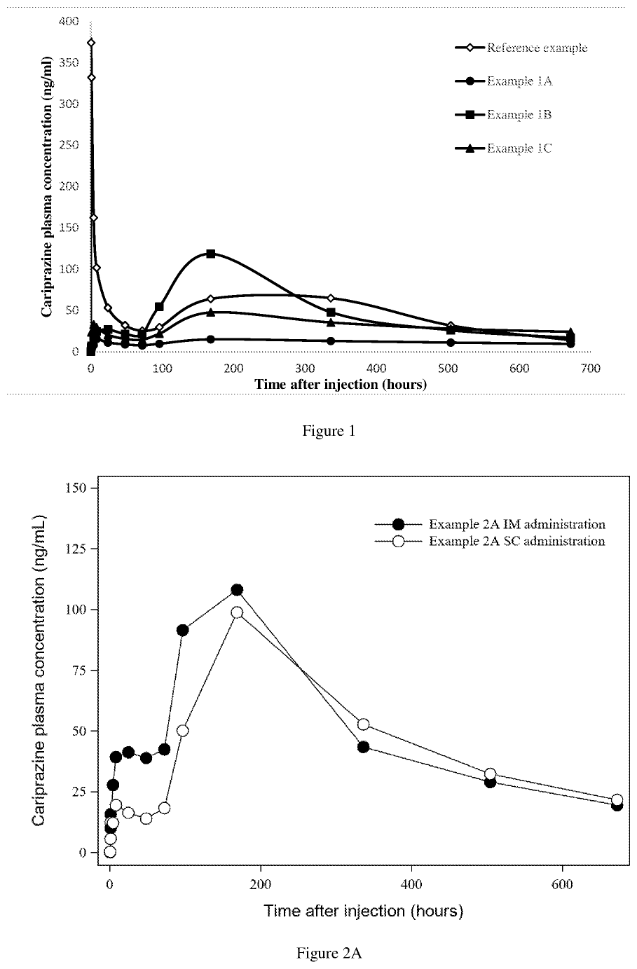

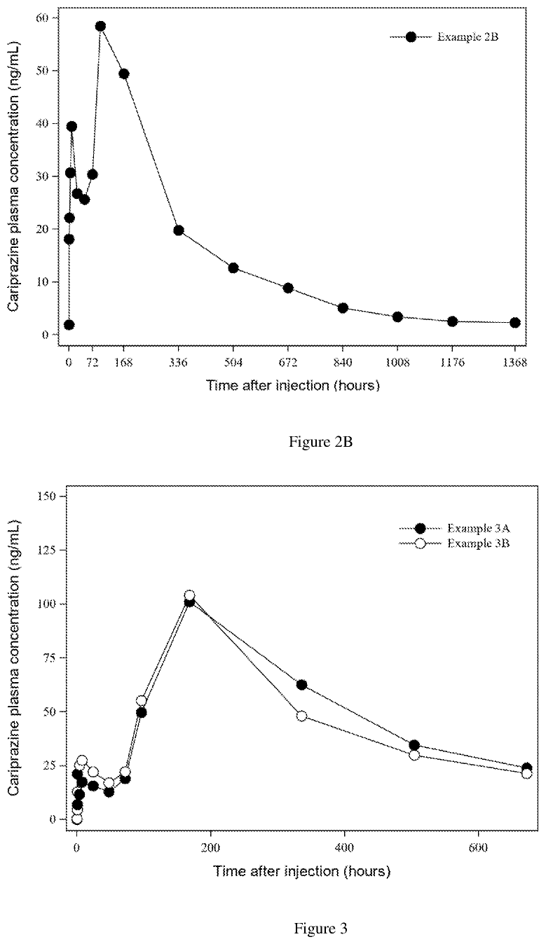

The controlled release formulation prepared in Example 2A was injected intramuscularly and subcutaneously to 6 rats, respectively, at doses of 100 mg/kg. Blood samples for the evaluation of systemic exposure were collected after 0, 0.5, 1, 4, 8, 24, 48, 72, 96, 168, 336, 504, and 672 hours of administration of the controlled release formulation and analyzed for Cariprazine. FIG. 2A shows the mean plasma concentrations vs. time profiles of Cariprazine in rats after intramuscular administration, and after subcutaneous administration, respectively.

The controlled release formulation prepared in Example 2B was injected into the thigh muscle of 8 rats, at doses of 45 mg/kg. Blood samples for the evaluation of systemic exposure were collected after 0, 0.5, 1, 4, 8, 24, 48, 72, 96, 168, 336, 504, 672, 840, 1008, 1176, and 1368 hours of administration of the controlled release formulation and analyzed for Cariprazine. FIG. 2B shows the mean plasma concentrations vs. time profiles of Cariprazine in rats after intramuscular administration.

The controlled release formulations prepared in Examples 3A and 3B were injected subcutaneously to 6 rats, respectively, at doses of 100 mg/kg. Blood samples for the evaluation of systemic exposure were collected after 0, 0.5, 1, 4, 8, 24, 48, 72, 96, 168, 336, 504, and 672 hours of administration of the controlled release formulation and analyzed for Cariprazine. FIG. 3 shows the mean plasma concentrations vs. time profiles of Cariprazine in rats after subcutaneous administration.

The controlled release injectable formulations showed steady serum concentration for at least one week, with a minimal burst effect.

Based on the results of the pharmacokinetic studies, it can be seen, that from the formulations of the present invention the concentrations of Cariprazine increase slowly after the administration of the injection, and the slow depot release maintains the Cariprazine concentration within the desired range.

Moreover, it can also be seen, that, in contrast to the formulations of the present invention comprising Cariprazine base, the formulation according to the reference example, comprising Cariprazine hydrochloride shows a remarkable burst effect, thus, it is therapeutically disadvantageous.

In addition, based on the results obtained in the in vivo test of the controlled release formulations of Example 3A and 3B, it can also be stated, that the amount of the wetting agent has no effect on the pharmacokinetic profile.

1. A controlled release injectable formulation in the form of a suspension, wherein upon injection, the formulation releases cariprazine over a period of at least one week, wherein the formulation comprises: cariprazine, one or more buffering agents, optionally one or more bulking agents, optionally one or more wetting agents, optionally one or more viscosity increasing agents, optionally one or more pH-adjusting agents, and an aqueous vehicle.

2. The formulation according to claim 1, containing cariprazine having a volume mean diameter within the range from about 1 to about 250 μm.

3. The formulation according to claim 1, containing cariprazine having a volume mean diameter within the range from about 3 to about 100 μm.

4. The formulation according to claim 1, containing cariprazine having a volume mean diameter within the range from about 3 to about 50 μm.

5. The formulation according to claim 1, where:
a) the cariprazine is present in an amount within the range from about 0.5 to about 25 weight/volume %,
b) the buffering agent is present in an amount within the range from about 0.1 to about 60 weight/volume %,
c) the bulking agent is present in an amount within the range from about 0 to about 5 weight/volume %,
d) the wetting agent is present in an amount within the range from about 0 to about 5 weight/volume %,
e) the viscosity increasing agent is present in an amount within the range from about 0 to about 15 weight/volume %,
f) the pH-adjusting agent is present in an amount within the range from about Oto about 3 weight/volume %,
based on the total volume of the suspension, and
g) wherein the aqueous vehicle is water for injections present in an amount of 0.5 to 4 ml.

6. A freeze-dried controlled release formulation, which comprises: cariprazine, one or more bulking agents, and/or one or more buffering agents, optionally one or more wetting agents, optionally one or more viscosity increasing agents, and optionally one or more pH-adjusting agents, which formulation upon constitution with an aqueous reconstitution medium forms an injectable formulation, which upon injection releases cariprazine over a period of at least one week.

7. The freeze-dried formulation according to claim 6, wherein the cariprazine has a volume mean diameter of about 1 to about 250 μm.

8. The formulation according to claim 6, wherein the cariprazine has a volume mean diameter within the range from about 3 to about 100 μm.

9. The formulation according to claim 6, wherein the cariprazine has a volume mean diameter from about 3 to about 50 μm.

10.-13. (canceled)

14. A method of treating a patient suffering from pathological conditions which require the modulation of dopamine receptors, selected from the group of psychoses, drug abuse, cognitive impairment accompanying schizophrenia, mild-to-moderate cognitive deficits, dementia, psychotic states associated with dementia, eating disorders, attention deficit disorders, hyperactivity disorders in children, psychotic depression, mania, paranoid and delusional disorders, dyskinetic disorders, anxiety, sexual dysfunction, sleep disorders, emesis, aggression and autism, wherein the method comprises the administration of the formulation according to claim 1 less frequent than daily to a patient in need thereof.

15. Method of treating a patient according to claim 14, wherein the patient is suffering from schizophrenia, and/or mania, and/or depression, and/or autism.

16. The method of claim 14, wherein the psychoses comprise schizophrenia and schizo-affective disorders.

17. The method of claim 14, wherein the cognitive impairment accompanying schizophrenia comprises positive symptoms, negative symptoms, and cognitive symptoms.
